(19) United States
(12) Patent Application Publication
(10) Pub. No.: US 2025/0248673 A1
(43) Pub. Date: Aug. 7, 2025

(54) METHOD AND SYSTEM FOR DETERMINING PRECISE DENTAL X-RAY ANGULATION BASED ON ANATOMICAL DENTAL FEATURES

(71) Applicant: Suzanne Cano, Clayton, NC (US)

(72) Inventor: Suzanne Cano, Clayton, NC (US)

(21) Appl. No.: 18/433,268

(22) Filed: Feb. 5, 2024

(51) Int. Cl. A61B 6/51; A61C 13/34; G06T 7/70; G06T 11/00

(52) U.S. Cl. CPC A61B 6/51; A61C 13/34; G06T 7/70; G06T 11/001; G06T 2207/30036; G06T 2210/41

(57) ABSTRACT

According to an aspect of the present invention, there is provided method of determining precise dental x-ray angulation based on the anatomical dental features of a patient, comprising: capturing one or more images of the teeth of the patient; sending the pictures will be sent to a server; customizing one or more x-ray angles based on algorithmic analysis of the images; displaying a tooth model of the patient on a display screen; representing, on the display screen, the teeth in an initial color; and updating the display to turn the corresponding teeth a final color to indicate completion as an x-ray angulation device achieves the customized angles.

### BACKGROUND

U.S. Pat. No. 10,905,386 to the present inventor disclosed a method for aiming a radiant energy projector for capturing one or more desired image(s) of a physiology of a patient, where the radiant energy is to be projected toward an object area of the physiology, the object area being an object of interest to be captured in the desired image(s), the method comprising: determining a plurality of reference points on the external surface of the physiology of the patient using computerized mapping, wherein each one of the reference points is laterally displaced from the object area relative to a direction of radiant energy to be projected; placing an energy responsive image forming medium proximate the object area of the physiology; projecting at least one indicator light beam fixed to the radiant energy projector, wherein the step includes projecting at least one indicator light beam onto the physiology of the patient to provide a general idea of the direction of the radiant energy projector toward the physiology of the patient; utilizing the computer generated plurality of reference points on the surface of the physiology of the patent to assess registration of the radiant energy projector, wherein registration of the radiant energy projector completed when the radiant energy projector is in proper orientation relative to the computer mapped reference points on the physiology of the patient; and operating the radiant energy projector to project radiant energy to generate the desired image(s) in the energy responsive image forming medium when the assessed registration between the radiant energy projector and the reference points mapped on the physiology of the patient meets a predetermined threshold of accuracy.

However, it would be desirable to further increase the accuracy of dental x-ray angulation, including by calculating the proper angle based on the patient's true dental anatomy.

### SUMMARY OF INVENTION

Therefore, the present invention provides systems and methods for customizing dental x-ray angulation leveraging Al algorithms. Images taken of a patient can be sent to the system of the present invention, and algorithms of the present invention can analyze the images taken of the patient to customize the proper dental x-ray angle based on the patient's calculated dental anatomy. Measuring the distance from the crown of the tooth to the apex of the tooth, the algorithms would calculate from the individual measurements from the picture using arbitrary degrees currently used for capturing a good diagnostic dental x-ray that are currently found in dental schools to determine angles needed to perform an approximation of the anatomy. This can be done for human and animal purposes.

According to an aspect of the present invention, there is provided method of determining precise dental x-ray angulation based on the anatomical dental features of a patient, comprising: capturing one or more images of the teeth of the patient; sending the pictures will be sent to a server; customizing one or more x-ray angles based on algorithmic analysis of the images; displaying a tooth model of the patient on a display screen; representing, on the display screen, the teeth in an initial color; and updating the display to turn the corresponding teeth a final color to indicate completion as an x-ray angulation device achieves the customized angles. Once the calculation has been performed the information would be sent back to the device, so that it would set itself to the customized angles for the individual patient. The end user will then be able to perform the single or series of x-rays needed in the automated or manual mode. The laser will auto set itself to the calculations based on the algorithms needed. The end user will have the customized angles in the device ready to acquire.

### BRIEF DESCRIPTION OF THE DRAWINGS

FIG. 1 illustrates a digital sensor.

FIG. 2 is an example of a mouth piece intended to hold the dental x-ray sensor in place.

FIG. 3 illustrates a dental x-ray machine with an automated dental x-ray positioner.

FIG. 4 illustrates an interface used in embodiments of the present invention.

FIG. 5 illustrates a flow chart for a process according to an embodiment of the present invention.

### DETAILED DESCRIPTION

The LunaLite Ai App and Software will be cloud-based, allowing for the storage and tracking of patients' dental data. Each patient's true angles will be saved for future visits. When the patient returns for additional x-rays, the end-user can retrieve the stored data, open the tooth model on the screen, and send the information to the LunaLite device for programming the specific angle required for the patient's dental anatomy.

The vision for LunaLite Ai is to continue developing and improving the angulation determination algorithms using the gathered data. By considering measurements such as crown-to-gingival distances, the software aims to calculate the apex position and determine precise angles based on established textbook angulation standards. The goal is to provide a cloud-based solution that offers customized angulation and saves patient data. Additionally, LunaLite Ai aims to collaborate with third-party Al companies to integrate their solutions and enhance the accuracy and efficiency of diagnostic dental x-rays. The mechanics of LunaLite Ai Software include a LunaLite app for various operating systems, LunaLite software for computers, and Bluetooth integration for communication with the LunaLite units.

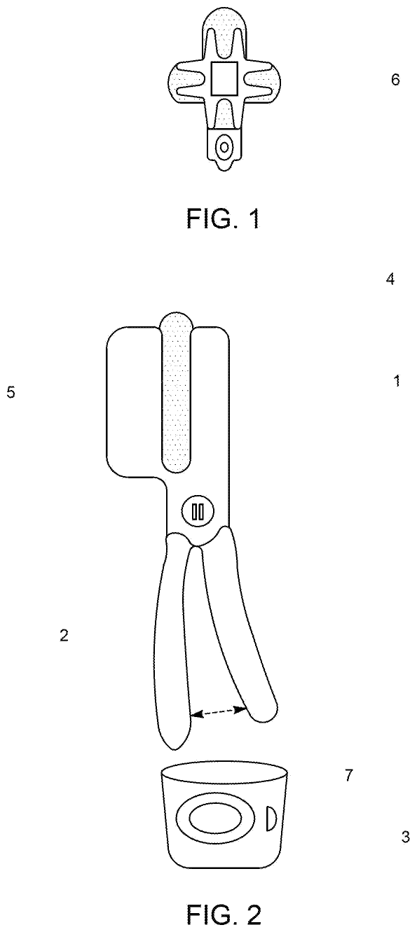

FIG. 1 illustrates a digital sensor.

The mouth piece has a sensor 6 at the end of the device to monitor patient movement on humans/animal. When movement occurs a light or a beep will alert the end-user of patent movement. This will allow the end-user to go back and reposition the mouth piece and the patient to minimize a retake or a missed dental x-ray shot.

The device can include a gyroscope, battery, breadboard, firmware, laser/laser host, code, beep sensor, on/off switch, USB connectivity, program screen, surgical conehead straps, and a rubber stopper at the bottom to prevent sliding.

A mouthpiece can be designed to hold digital sensors, film, or phosphor plates. It can feature an illuminating or removable fluorescent light at the tip to detect movement and be able to charge through USB. When movement occurs, the light will turn on, indicating the need for readjustment. The mouthpieces can be autoclavable and available in different sizes to accommodate patients of varying sizes. They can also feature markers (dots or lines) to assist with sensor positioning (centering).

By combining the LunaLite automated dental x-ray positioner with Al algorithms and a cloud-based software solution, LunaLite Ai aims to revolutionize dental x-ray angulation, improving accuracy, efficiency, and patient experience.

FIG. 2 is an example of a mouth piece intended to hold the dental x-ray sensor in place.

Rubbery material 4 holds the sensor in place.

Upper bite plate 1 and lower bite place 5 is configured for a patient to bite down.

Handles 2 can be used by the operator to adjust the mouthpiece.

LED light 3 is configured to light up to indicate a movement of the sensor device.

USB-C 7 is for charging the device or accessing data, for example.

A user, typically an assistant or hygienist, will capture three pictures of the patient's midline and side teeth using an ortho retractor. Cheek retractors are important for keeping the soft tissues away from the teeth and allowing easy camera viewing of the dentition. Generally, these come in plastic or stainless steel, although plastic retractors are may be more comfortable for patients.

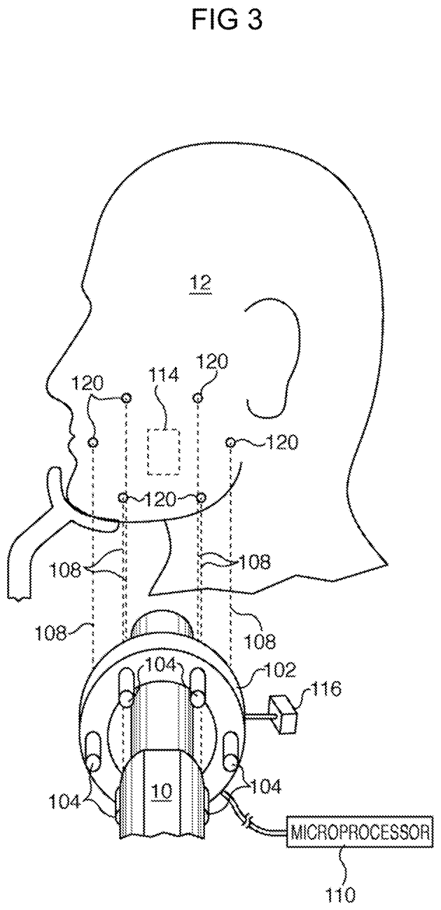

FIG. 3 illustrates a dental x-ray machine with an automated dental x-ray positioner.

The automated dental x-ray positioner, guided by a laser or beep, replaces manual aligners like Rinn holders or XCPs. This device simplifies the positioning process, allowing for more precise angles and automated movement through programmed angles for a full-mouth series (FMX) in a 30-second intervals between each angle timeframe. The device features a non-slip back, surgical conehead strap to securely hold the unit in place, and a black button on top to select the desired dental angle. The device can be used FMX, bitewing (BW) and periapical (PA) x-rays. The device can have a 10-degree variance when the laser was on, providing flexibility for patients of different sizes.

Apparatus 100 is provided for aiming a radiant energy projector 10 for capturing one or more desired images of a physiology of a patient 12 using radiant energy projector 10. Apparatus 100 may comprise a chassis 102 including a plurality of lighting projectors 104, and a mounting 106 enabling securing of chassis 102 on radiant energy projector 10 such that lighting projectors 104 are aimed parallel to an axis 108 of radiant energy projected from radiant energy projector 10.

Apparatus 100 may also comprise a microprocessor 110 having computer instructions loaded thereinto for comparing positional data of radiant energy projector 10 to predetermined positional data of patient 12, and to determine alignment of radiant energy projector 10 with patient 12, and an automated indicator actuatable by microprocessor 110 to indicate when the positional data of radiant energy projector 10 is aligned with patient 12. Optionally, apparatus 100 may comprise a holder 112 operable to secure the physiology of patient 12 in a constant location relative to radiant energy projector 10. Holder 112 is optional because there are ways to determine alignment of radiant energy projector 10 with patient 12 other than those utilizing holder 12, as will be further explained hereinafter.

Radiant energy projector 10 may be an X-ray emitter or a CT signal emitter for example. A desired image is that image which captures a specific targeted portion of the physiology for dental or medical analysis, such as an X-ray image of a tooth site. Chassis 102 may be a ring as depicted herein, or any other rigid structure capable of holding lighting projectors 104 in a position such that lighting projectors 104 will emit light in an array such as that indicated by axes 108 of projected light.

Mounting 106 is depicted as an arm attached to chassis 102 and to radiant energy projector 10, but may take other forms. Illustratively, mounting 106 may comprise clips, clamps, threaded fasteners, friction fit and/or other devices capable of holding chassis 102 to radiant energy projector 10 in a constant orientation or position.

The automated indicator may be a discrete element unto itself (this option is not shown), or alternatively, may be realized by manipulating lighting characteristics of light produced by lighting projectors 104, as will be described hereinafter.

Holder 112 is depicted as a chin support in FIG. 1, but may take other forms. Any device which supports, holds, or restrains patient 12 in a suitably fixed location for capturing the desired image will suffice. It will be recognized that the desired image will be produced by an energy responsive image forming medium 114, such as X-ray film placed in the mouth of patient 12 in conventional manner. The computer instructions may be based on a predetermined geometric relationship between radiant energy projector 10 and a patient 12 held in holder 112. That is, distance and azimuthal orientation of radiant energy projector 10 and patient 12 in holder 112 may be determined beforehand and loaded into memory (not separately shown) of microprocessor 110. For example, such instructions may be based upon entered data of the patient 12 based at least in part on sex, age, simple measurements, and/or any other form of data of the patient 12.

Alternatively, the computer instructions may be based on an image of the physiology of patient 12. In this case, an image of the physiology of patient 10 is captured, either by a camera integrated with apparatus 100 or separate therefrom. The resulting image, as a digital photograph or 3D scan, or possibly a CT scan, would yield data in the form of a data plot or topography plot. This data would then be processed to generate a signal annunciating acceptable positioning of radiant energy projector 10, as will be further explained hereinafter. Hence the computer instructions may include data corresponding to at least simple data of a patient (age, sex, simple measurements, etc.) or more precise data plots, such as, topography plots which may be derived from the image or scan of the physiology of patient 12.

Microprocessor 110 is shown connected to chassis 102 by a cable 118. Apparatus 100 will be understood to include necessary power source, power and control circuitry, and operator controls (none of these elements are shown) to arrive at a system functional as described.

It is possible that apparatus 100 may further comprise an image capture device 116 in communication with microprocessor 110, whereby apparatus 100 can obtain the image of the physiology of patient 12 independently of a separate camera or other image capture device such as a cellular telephone (none of these options is shown).

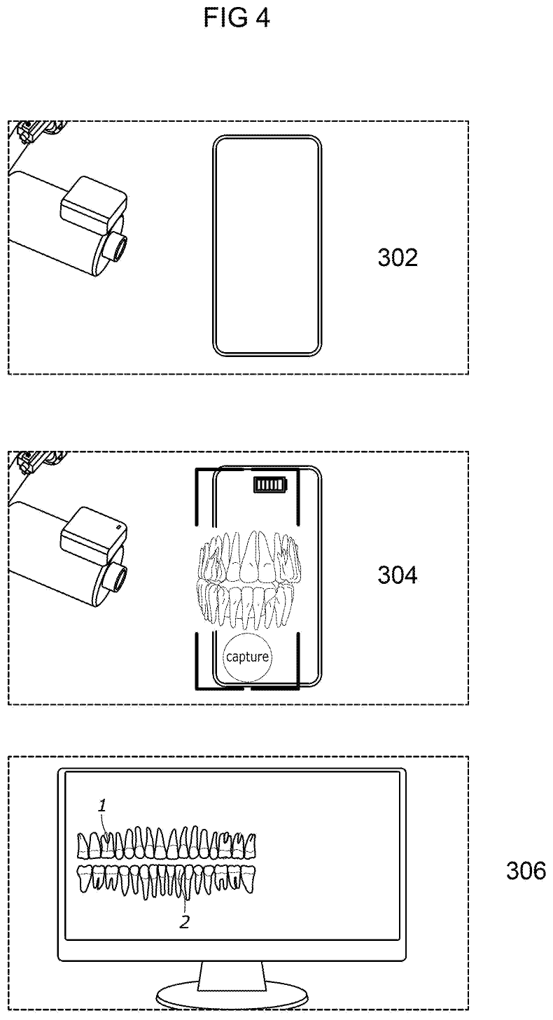

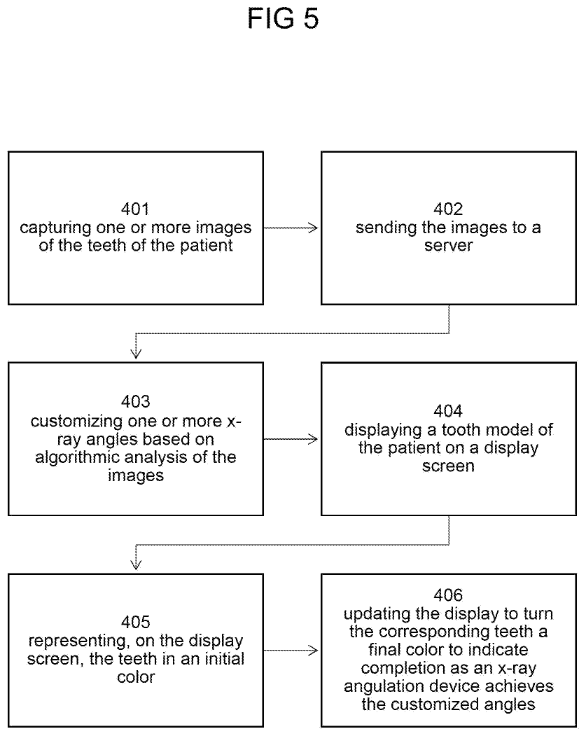

FIG. 4 illustrates an interface used in embodiments of the present invention.

The LunaLite Ai App and Software 302 will be available for download on computers and mobile devices.

The software will feature a patient database with names and tooth models. When performing x-rays on a patient, the end-user can select the patient's name, view the tooth model on the screen 304, and see the teeth represented as green when the x-rays have been calculated and the device has received the customized angles needed, the device (LunaLite) has received the proper angulations.

As the LunaLite device achieves the programmed angles, the corresponding teeth on the software will turn red to indicate completion. This visual feedback 306 will help the end-user position the dental x-ray machine accurately.

FIG. 4 illustrates a flow chart for a process according to an embodiment of the present invention.

Step 401 is capturing one or more images of the teeth of the patient.

Step 402 is sending the images to a server.

Step 403 is customizing one or more x-ray angles based on algorithmic analysis of the images.

Step 404 is displaying a tooth model of the patient on a display screen.

Step 405 is representing, on the display screen, the teeth in an initial color.

Step 406 is updating the display to turn the corresponding teeth a final color to indicate completion as an x-ray angulation device achieves the customized angles.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Similar numerals designate similar elements among the several figures. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A method of determining precise dental x-ray angulation based on the anatomical dental features of a patient, comprising:
capturing one or more images of the teeth of the patient;
sending the images to a server;
customizing one or more x-ray angles based on algorithmic analysis of the images;
displaying a tooth model of the patient on a display screen;
representing, on the display screen, the teeth in an initial color; and
updating the display to turn the corresponding teeth a final color to indicate completion as an x-ray angulation device achieves the customized angles.

2. The method of determining precise dental x-ray angulation based on the anatomical dental features of a patient of claim 1, further comprising saving the customized angles on a computer memory device for future visits.

3. The method of determining precise dental x-ray angulation based on the anatomical dental features of a patient of claim 2, further comprising retrieving the saved customized angles, displaying the tooth model on the screen, and sending the information to the x-ray angulation device.

4. The method of determining precise dental x-ray angulation based on the anatomical dental features of a patient of claim 1, wherein the x-ray angulation is angulation for full-mouth (FMX) x-rays.

5. The method of determining precise dental x-ray angulation based on the anatomical dental features of a patient of claim 1, wherein the x-ray angulation is angulation for bitewing (BW) x-rays.

6. The method of determining precise dental x-ray angulation based on the anatomical dental features of a patient of claim 1, wherein the x-ray angulation is angulation for periapical (PA) x-rays.

7. The method of determining precise dental x-ray angulation based on the anatomical dental features of a patient of claim 1, further comprising sending feedback regarding the x-ray quality obtained from the x-ray image data captured using the customized angles to the server.

8. The method of determining precise dental x-ray angulation based on the anatomical dental features of a patient of claim 7, further comprising developing and improving the angulation determination algorithms using the images sent to the server and feedback regarding the x-ray quality obtained from the x-ray image data captured using the customized angles.
